SEQUENCE LISTING (8 sequences)

SEQ ID NO: 1 - DNA, 26 bp, Artificial
  Primer Sequence
gcatgctgtagagcaagaaatggagc

SEQ ID NO: 2 - DNA, 21 bp, Artificial
  Primer Sequence
gctcccaagaacccaaggaac

SEQ ID NO: 3 - DNA, 28 bp, Artificial
  Primer Sequence
cgattcatcttttttctctttgcactgt

SEQ ID NO: 4 - DNA, 33 bp, Artificial
  Primer Sequence
tagagccctggaagcatccaggaagtcagccta

SEQ ID NO: 5 - DNA, 23 bp, Artificial
  Primer Sequence
catggtagaacagatgcatgagg

SEQ ID NO: 6 - DNA, 26 bp, Artificial
  Primer Sequence
tccagtccccccttttcttttaaaaa

SEQ ID NO: 7 - DNA, 37 bp, Artificial
  Primer Sequence
ggcggcggcatcgattcacaagagagtgagctcaagc

SEQ ID NO: 8 - DNA, 37 bp, Artificial
  Primer Sequence
gcggcggcggctagcacagaaaaattgtgggtcacag

(19) United States
(12) Patent Application Publication
(10) Pub. No.: US 2008/0267993 A1
(43) Pub. Date: Oct. 30, 2008

(54) AIDS vaccines

(75) Inventors: Nancy L. Haigwood, Seattle, WA (US); Wendy Blay, Seattle, WA (US); Leonidas Stamatatos, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: Seattle Biomedical Research Institute, Seattle, WA (US); University of Washington, Seattle, WA (US)

(21) Appl. No.: 11/713,124

(22) Filed: Mar. 2, 2007

Related U.S. Application Data
(63) Continuation of application No. 11/096,698, filed on Mar. 31, 2005, now abandoned.
(60) Provisional application No. 60/558,181, filed on Mar. 31, 2004.

(51) Int. Cl. A61K 39/295; A61P 31/18

(52) U.S. Cl. 424/202.1

(57) ABSTRACT

The invention provides vaccination protocols for administering immunogens to a primate host in order to promote the formation of neutralizing antibodies (NAbs) against primate immunodeficiency viruses. In some embodiments, the vaccination protocols comprise the step of administering to a primate host a first immunogen comprising at least one primate immunodeficiency virus Envelope (env) sequence having a first set of consensus glycosylation sequences, followed by a second immunogen comprising at least one primate immunodeficiency virus env sequence having a second set of consensus glycosylation sequences, wherein the differences between the first set of consensus glycosylation sequences and the second set of consensus glycosylation sequences comprise differences in consensus glycosylation sequences observed in HIV isolates obtained at different time points of a natural infection.

### CROSS-REFERENCE TO RELATED APPLICATION

This application is a continuation of U.S. application Ser. No. 11/096,698, filed Mar. 31, 2005, which claims the benefit of U.S. Provisional Application No. 60/558,181, filed Mar. 31, 2004.

### STATEMENT OF GOVERNMENT LICENSE RIGHTS

The U.S. Government has a paid-up license in this invention and the right in limited circumstances to require the patent owner to license others on reasonable terms as provided for by the terms of Grant No. P01 A1-26503 awarded by the National Institutes of Health.

### FIELD OF THE INVENTION

The invention relates to HIV vaccines, and particularly to improved immunogens for generating HIV neutralizing antibodies.

### BACKGROUND OF THE INVENTION

Vaccine development against HIV has been hampered by the extensive diversity and elaborate escape mechanisms of the Envelope protein (Env). Env is the most variable of the HIV genes, typically diversifying in the range of 1% per year in patients. Mutations resulting in amino acid changes have been shown to contribute to escape from cytotoxic T lymphocytes (CTL) as well as neutralizing antibodies (NAbs). Because Env engages both the CD4 receptor and chemokine coreceptors for binding and entry, mutations to regions involved in these activities can result in altered tropism. Early focus on the hypervariable regions showed the importance of these hydrophilic loops in generating NAbs (Haigwood et al. (1990) AIDS Res. Hum. Retroviruses 6:855-69; Putney et al. (1986) Science 234:1392-5). HIV is one of the most heavily glycosylated proteins currently known, with glycans comprising more than 50% of the molecular weight of Env. These glycans play an important role in enabling the virus to escape neutralizing antibodies (NAbs) (Burns et al. (1993) J. Virol. 67:4104-12; Rudensey et al. (1998) J. Virol. 72:209-16). Recently Wei et al. (Wei et al. (2003) Nature 422:307-12) proposed a model wherein the carbohydrate moieties of HIV comprise a glycan shield that creates a fluid canopy around the conserved neutralizing epitopes of the protein domain beneath. This shield is able to evolve in response to neutralizing antibodies (NAbs), creating a continuously changing landscape on the viral Envelope. Functionality of the Env protein appears to limit the number of potential N-linked glycosylation (PNG) sites, resulting in maintenance of this range of glycans and redistribution of their placement.

A major biological question that remains poorly understood concerns the relationship between the adaptive immune response and Env mutations. Neutralizing antibody (NAb) titers correlate with lack of disease progression in HIV-infected long-term nonprogressors (Cao et al. (1995) N. Engl. J. Med. 332:201-8), and viral persistence may depend on the selection of NAb escape variants (Ciurea et al. (2000) Proc. Natl. Acad. Sci. U.S.A. 97:2749-54; Wei et al. (2003) Nature 422:307-12). Eventually, in some patients, the NAb response can show evidence of affinity maturation and recognition of common determinants conserved among sequentially distinct isolates (Steimer et al. (1991) Science 254:105-8). However, this maturation is a multi-year process and it is not universal, occurring in a minority of patients (Moog et al. (1997) J. Virol. 71:3734-41).

Thus, there is a need for improved vaccination protocols for generating HIV neutralizing antibodies. The present invention addresses this need and others.

### SUMMARY OF THE INVENTION

One aspect of the invention provides vaccination protocols for administering immunogens to a primate host in order to promote the formation of neutralizing antibodies (NAbs) against primate immunodeficiency viruses. In some embodiments, the vaccination protocols comprise the step of administering to a primate host a first immunogen comprising at least one primate immunodeficiency virus Envelope (env) sequence having a first set of consensus glycosylation sequences, followed by a second immunogen comprising at least one primate immunodeficiency virus env sequence having a second set of consensus glycosylation sequences, wherein the differences between the first set of consensus glycosylation sequences and the second set of consensus glycosylation sequences comprise differences in consensus glycosylation sequences observed in HIV isolates obtained at different time points of a natural infection. For example, the primate immunodeficiency virus may be Human Immunodeficiency Virus type 1 and the primate host may be a human host.

In some vaccination protocols, the differences between the first set of consensus glycosylation sequences and the second set of consensus glycosylation sequences comprise one or more consensus N-linked glycosylation sequence changes in at least one of V1, V2, C2, V4, and V5. For example, the differences between the first set of consensus glycosylation sequences and the second set of consensus glycosylation sequences comprise at least one of:

(a) an addition of a consensus N-linked glycosylation sequence in V1 at a position corresponding to position 143 or 144 in the HIV-89.6 env sequence;

(b) a shift of a consensus N-linked glycosylation sequence in V2 from a position corresponding to position 186 in the HIV-89.6 env sequence to a position corresponding to position 188 in the HIV-89.6 env sequence;

(c) an addition of a consensus N-linked glycosylation sequence addition in C2 at a position corresponding to position 276 in the HIV-89.6 env sequence;

(d) an addition of a consensus N-linked glycosylation sequence addition in V4 at a position corresponding to position 386 in the HIV-89.6 env sequence;

(e) an addition of a consensus N-linked glycosylation sequence addition in C2 at a position corresponding to position 397 in the HIV-89.6 env sequence; and

(f) a shift of a consensus N-linked glycosylation sequence in V5 from a position corresponding to position 460 in the HIV-89.6 env sequence to a position corresponding to position 462 or 463 in the HIV-89.6 env sequence.

The first immunogen and/or the second immunogen may comprise a single env sequence or may comprise multiple env sequences.

In some embodiments, there are additional differences between the first and the second immunogens, for example, an increase in acidity in V1V2, and/or the addition of a proline in V5.

In some embodiments, the vaccination protocols of the invention are used as therapeutic vaccines in primate hosts that are already infected with one or more primate immunodeficiency viruses.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a phylogenetic analysis of individual HIV-1 Envelope gp120 proviral clones derived from different macaques infected with SHIV-89.6P at 32 weeks post-infection. The Maximum Likelihood tree is rooted on the consensus of the inoculum. Each macaque is denoted by a different symbol in the key. Seven animals form independent clusters while four animals diverge little from the inoculum.

FIG. 2 shows a graphical representation of the conservation of potential N-linked glycosylation sites (PNGs) in the deduced amino acid sequence of clones of Env from macaques infected with SHIV-89.6P using a program termed N-glycosite (www.hiv.lanl.gov/content/hiv-db/GLYCOSITE/glycosite.html) from the Los Alamos National Laboratory (LANL). One hundred two gp120 DNA clones were analyzed from eleven different SHIV-infected macaques, as in FIG. 1. The majority of PNGs are well conserved. The numbers of clones that are predicted to have a given PNG are shown as a fraction of the total on the Y axis. Each number on the X-axis corresponds to a PNG motif that is present in over 90% of the SHIV-89.6P clones analyzed. Sites denoted with (*) are also present in over 90% of the HIV-1 gp160 sequences analyzed from LANL. Grey lines at the top represent regions identified as hotspots of PNG variation based on HIV-1 gp160 published in the LANL HIV-1 database. Variable regions of the HIV-1 Envelope protein are shown as boxes.

FIG. 3 shows PNG combinations within the Env quasispecies members of each macaque at 32 weeks post-infection. Each horizontal bar corresponds to a combination of PNG changes that exist within the quasispecies of each animal. The number of clones with a given combination is shown to the left of the bars. The position of each PNG is shown along the top. If the PNG is present, it is shown as a vertical line.

FIG. 4 shows the accumulation of Env sequence changes over time in two macaques. Representative longitudinal sequences from two macaques with significant diversification are shown. The consensus sequence of Env in the SHIV-89.6P inoculum is shown along the top. Key regions of Env are that are discontinuous in the protein are shown as fused here to highlight the regions of interest. Four PNG additions (one each in V1, C2, two in V4) and two PNG shifts (V2 and V5) are shown. The region of V2 that undergoes a charge change and the proline addition in V5 are shown.

FIG. 5 shows that the net charge of V2 region becomes more acidic in animals with diversified quasispecies. A forty residue region from 152-192 has a median charge of plus 4 (+4) in the inoculum. The four macaques that do not diversify (K97107, K98099, L97191, and J97168) maintain this charge. The seven animals that diversify from the inoculum (A98069, T98098, J97156, K97246, L98152, T98108, and J98071) have mutations in this region that result in a more acidic (or more negative) net charge. The vertical line divides animals that do not have any significant PNG changes from those that do. Macaque numbers on the X axis are arrayed in are order by increasing levels of divergence.

FIG. 6 shows the development of homologous NAbs over time. Sera from the SHIV-89.6P infected macaques were tested against pseudotyped SHIV-89.6P in a Tzm-bl assay. The reciprocal titer for 50% neutralization is shown. The Y-axis for animal K98099 is 10-fold higher than the others. Each sample was run in triplicate and most data points represent the geometric mean of three independent assays. Macaques K97107, L97191, J97168, and L98152 have no detectable NAbs at any time point tested and are not shown.

### DETAILED DESCRIPTION OF THE PREFERRED EMBODIMENT

In one aspect, the invention provides vaccination protocols that promote the formation of neutralizing antibodies (NAbs) against primate immunodeficiency viruses such as Human Immunodeficiency Virus type I (HIV-1). In some embodiments, the vaccination protocol comprises administering to a primate host a first immunogen comprising at least one primate immunodeficiency virus Envelope (env) sequence having a first set of consensus glycosylation sequences, followed by a second immunogen comprising at least one primate immunodeficiency virus env sequence having a second set of consensus glycosylation sequences, wherein the differences between the first set of consensus glycosylation sequences and the second set of consensus glycosylation sequences comprise differences in consensus glycosylation sequences observed in HIV isolates obtained at different time points of a natural infection.

As used herein, the term “primate” refers to human and nonhuman primates. The term “primate immunodeficiency virus” includes human viruses, such as Human Immunodeficiency Virus type 1 (HIV-1) and Human Immunodeficiency Virus type 2 (HIV-2), as well as simian immunodeficiency viruses, including SIVcpz (chimpanzee) and SIV from the various Macaca species. Interviral recombinants with chimeric env genes are also included in this collection. The term “env” is used to describe the Envelope gene, and the term “Env” or “Envelope” is used to describe the translated protein sequence. In some embodiments of the invention, the primate immunodeficiency virus is HIV-1. The deduced amino acid sequence in each of the primate lentivirus members characterized to date has a typical length for each virus. The Env proteins from all of the primate lentivirus family members have common features, although their sequence homology may vary considerably. The gp120 protein is expressed as a gp160 precursor that is processed into gp120 (surface protein or SU) and gp41 (transmembrane protein or TM). Each gp120 protein is organized with more constant (C) and more variable (V) regions, where V regions are typically but not always bounded by highly conserved Cysteine (Cys) residues. The organization of the gp120 protein follows the pattern C1, V1-V2, C2, V3, C3, V4, C4, V5, C5. The locations of homologous regions can be inferred by aligning the predicted proteins. The conservation of these conserved and variable regions is likely to be constrained by functionality, as each of these proteins utilizes the CD4 or CD4 homolog for binding and cell entry, along with coreceptors that can be unique for each host. Therefore the CD4 binding site (CD4bs) is conserved, despite significant sequence divergence among the primate lentivirus family member env genes.

The term “primate host” refers to the host that is infectable by the primate immunodeficiency virus. According to the vaccination protocol of the invention, the primate host may already be infected by one or more primate immunodeficiency viruses before administering an immunogen, or it may be uninfected after administering one or both immunogens. Post-infection administration of immunogens (i.e., therapeutic vaccines) may accelerate the development of high avidity NAbs to control infection and make the host less susceptible to super-infection with related viruses.

The term “consensus glycosylation sequence” and refers to a potential glycosylation site (PGS), such as a potential N-linked glycosylation site (PNG, or consensus N-linked glycosylation site). The canonical PNG motif is Asn-X-Ser/Thr, where X represents any residue other than proline.

It has been observed that the primate lentiviruses mutate within each host and form a group of closely related sequences known as a “quasispecies.” The relationship between the adaptive immune response and Env mutations found in the individual quasispecies can be addressed in part by comparing the sequences within different individuals over time during disease progression and when there are alterations in Env functionality such as coreceptor usage. Such a comparison was performed using published sequences found in the Los Alamos National Laboratory (LANL) HIV Database. An analysis of 49 published full-length HIV-1 gp160 sequences in the LANL database revealed that approximately half of these glycans are in conserved regions (Zhang et al. (2004) Glycobiol. 14:1229-46). Of those that are not conserved, there appear to be small windows or “hotspots” where the glycans can vary as coreceptor switching occurs. However, this type of analysis does not directly link the Env mutations to the development of antibody or CTL responses. Comparisons of the locations of glycans in the primate lentivirus family members shows that they are remarkably conserved. The fraction of sequences with an N-linked glycosylation site at specific positions in the primate lentivirus Env predicted proteins varies among members that have been tested, but detailed longitudinal analyses have not been performed to determine how these patterns and frequencies change during infection in individuals.

Another approach to addressing this question is to determine whether key changes are similar in different individuals infected with the same virus. Using molecularly cloned or low diversity virus isolates to infect nonhuman primates, it is possible to monitor the fate of viral sequences over months or years post-infection in multiple individuals with a greater or lesser degree of control of viremia. These types of studies have been valuable in understanding viral mutations leading to immune escape from cytotoxic T cells (Watkins Tat escape data). Much of the data available from the analysis of mutations in Env in nonhuman primates has utilized SIV, and it has been shown that changes to PNGs can directly affect both (1) resistance to neutralization by sera from the infected macaques, and (2) pathogenicity of the virus itself. SIV is closely related to HIV-2 and differs significantly in Env from HIV-1; SIV and HIV-1 share no known neutralization determinants. Thus, specific changes in amino acids that are influenced by NAb pressure would be difficult to compare directly with HIV-1 in humans. In contrast, the chimeric SHIV viruses bearing HIV env genes have the potential for comparison with HIV env genes in their native context.

As described in EXAMPLE 1, we performed an analysis of eleven macaques that were infected with SHIV-89.6P for approximately one year. SHIV-89.6 was constructed with the HIV-89.6 env gene. The resulting in vivo passaged pathogenic SHIV-89.6P utilizes both CCR5 and CXCR4 as coreceptors, like the parent HIV-89.6. We observed a remarkable degree of conservation in patterns of change to Env PNG and alterations in V2 and V5 in all seven macaques with divergent virus, despite variation in overall amino acid sequences that was unique to each macaque. Data from this study suggests that the glycan shield and certain other key regions of Env may be under considerable constraint during viral divergence.

The Envelope protein of HIV-1 possesses an elaborate network of sugar moieties that have been recently termed the “glycan shield.” Prior work has shown the importance of Env glycosylation in enabling the virus to escape neutralizing antibodies (NAbs). We have used SHIV-89.6P infection of macaques from a vaccine study to determine whether specific patterns of potential N-linked glycosylation (PNG) develop over time, and how changes are related to host immune responses. Analysis of proviral env gp120 sequences obtained from eleven macaques over one year of infection demonstrated that Env from seven of the animals underwent remarkably similar patterns of change in loss, addition or shift in locations of PNG in this time frame. The total number of sites did not increase significantly, and of the 19 of the 23 PNGs present in the inoculum were conserved in the sequences from all macaques. The locations of 11 were rarely altered in each individual, including four PNGs located in hypervariable regions. These more conserved glycans are in positions that are predicted to lie on the silent face of Env. Surprisingly, we observed statistically significant variation in PNGs occurring within a small number of constrained regions at specific sites. These include additions on V1 and the N terminal side of V4 and PNG shifts on V2 and V5. Viruses from several of the macaques also add a PNG to the outer region of C2, an area identified as part of the CD4 binding site. The temporal development of changes differs in individual macaques, and linkages between changes can be studied by this method. Calculation of dN/dS ratios suggests that these changes are the result of selective pressure. In many of the highly diverged Envs from several macaques, we also observed charge change in V1V2 resulting in a net acidic charge, and a proline addition in V5. There is currently little evidence of heterologous NAbs prior to 1-year post infection in any of the animals in this study. We have observed convergent PNG changes that correlate with stronger homologous NAbs and they may be correlated with stronger heterologous NAbs.

In the vaccination protocols of the invention, the differences between the first and second set of consensus glycosylation sequences may reflect the differences observed in the consensus glycosylation sequences in viral isolates obtained at earlier and later time points of a natural infection. Thus, the first set of consensus glycosylation sequences may resemble the pattern of consensus glycosylation sequences in viral isolates obtained at an earlier stage of a natural infection and the second set of consensus glycosylation sequences may resemble the pattern of consensus glycosylation sequences in viral isolates obtained at a later stage of a natural infection. In some embodiments, the differences between the first set of consensus glycosylation sequences and the second set of consensus glycosylation sequences comprises the addition and/or deletion of one or more consensus glycosylation sequences.

The differences between the first set of consensus glycosylation sequences and the second set of consensus glycosylation sequences may comprise one or more consensus N-linked glycosylation sequence changes in at least one of V1, V2, C2, V4, and V5. In some embodiments, one or more consensus N-linked glycosylation sequence changes comprises a consensus N-linked glycosylation sequence addition in V1, for example at a position corresponding to position 143 or 144 in the HIV-89.6 env sequence.

In some embodiments, one or more consensus N-linked glycosylation sequence changes comprises a consensus N-linked glycosylation sequence addition in V2, for example at a position corresponding to position 188 in the HIV-89.6 env sequence. In some embodiments, one or more consensus N-linked glycosylation sequence changes comprises a consensus N-linked glycosylation sequence deletion in V2, for example at a position corresponding to position 186 in the HIV-89.6 env sequence. In some embodiments, one or more consensus N-linked glycosylation sequence changes comprises a consensus N-linked glycosylation sequence shift in V2, for example a shift from a position corresponding to position 186 in the HIV-89.6 env sequence to a position corresponding to position 188 in the HIV-89.6 env sequence.

In some embodiments, one or more consensus N-linked glycosylation sequence changes comprises a consensus N-linked glycosylation sequence addition in C2, for example at a position corresponding to position 276 in the HIV-89.6 env sequence. In some embodiments, one or more consensus N-linked glycosylation sequence changes comprises a consensus N-linked glycosylation sequence addition in V4, for example at a position corresponding to position 386 in the HIV-89.6 env sequence, or at a position corresponding to position 397 in the HIV-89.6 env sequence.

In some embodiments, one or more consensus N-linked glycosylation sequence changes comprises a consensus N-linked glycosylation sequence addition in V5, for example, at a position corresponding to position 462 or 463 in the HIV-89.6 env sequence. In some embodiments, one or more consensus N-linked glycosylation sequence changes comprises a consensus N-linked glycosylation sequence deletion in V5, for example, at a position corresponding to position 460 in the HIV-89.6 env sequence. In some embodiments, one or more consensus N-linked glycosylation sequence changes comprises a consensus N-linked glycosylation sequence shift in V5, for example, a shift from a position corresponding to position 460 in the HIV-89.6 env sequence to a position corresponding to position 462 or 463 in the HIV-89.6 env sequence.

In some embodiments of the vaccination protocols of the invention, there are additional differences between the first and the second immunogens, for example, an increase in acidity in V1V2, and/or the addition of a proline in V5 at a position corresponding to position 462 or 463 in the HIV-89.6 env sequence, as described in EXAMPLE 1.

Useful vaccination protocols may include recombinant vaccines that express the Env protein. Examples of this include recombinant viruses such as poxviruses (vaccinia virus, modified Vaccinia Ankara, and canarypox vectors); recombinant adenoviruses that are replication competent or replication incompetent; recombinant adenovirus-associated virus; Venezuelan Equine Encephalitis viruses; or Vesicular Stomatitis Viruses, for example. Additional vaccines may include DNA expression vectors typically based on the human cytomegalovirus Immediate Early-I promoter and competent to direct Env expression in mammalian cells (“DNA vaccines”) that may be delivered by a variety of routes (intramuscular, intradermal, transdermal, oral, intravaginal, intrarectal, intranasal, etc.). Further, vaccines may consist of recombinant proteins that are delivered in adjuvants or in microspherical biodegradable particles to various sites using intramuscular or intradermal or oral immunization. These vaccines may be used alone or in combination with one another. Vaccination with Env may be accompanied by vaccination with other HIV proteins or other viral proteins at the same time using similar or identical vectors or systems to achieve responses to Env and the other proteins at the same time. Oligomeric forms of the HIV-1 Env protein may be used.

Sequential delivery of vaccines for humans usually follows the time course of vaccination at week 0, week 4 (1 month) and week 24-26 (6 months). This time period of sequential immunization is adequate and may be improved by additional immunizations at 10 months and 12 months, for example. It will be appreciated that the time schedules for immunization may be varied and that many possible time schedules for immunization will be effective.

In some embodiments, env gene expression vectors are used as DNA vaccines, as was performed in the publication (Doria-Rose et al. (2003) J. Virol. 77:11563-77). The DNA vectors expressing individual Env proteins may be designed as codon-optimized genes for higher level expression and better immuongenicity in vivo (Barouch et al. (2000) Proc. Natl. Acad. Sci. U.S.A. 97:4192-7). Individual env gene expression plasmids and combinations of these plasmids can be delivered by a variety of methods, including intramuscular injection with or without adjuvants such as bupivicaine using a needle or a Biojector device, or by intradermal inoculation with a needle. Multiple immunizations of DNA can be delivered without any adverse effects upon the recipient, and there is no anti-DNA response to limit the use of 10 or more injections of DNA. Following multiple DNA priming, the immune responses can be boosted with recombinant viral vectors expressing one or more env genes, or else recombinant purified oligomeric gp140 or gp160 Envelope protein combined with adjuvant can be used. Alternatively, recombinant viral vectors (e.g. adenovirus or attenuated vaccinia viruses, such as Modified Vaccinia Ankara or MVA) can be used alone or in combination with DNA immunizations (before or after) to prime and boost, with or without additional Env protein boosting. Exemplary vaccination protocols are provided in EXAMPLES 2 and 3.

Success of the vaccination protocol can be measured by showing that sera or plasma from immunized test animals have antiviral Neutralizing Antibodies (associated with IgA, secretory IgA, or IgG) effective in blocking viral infection in vitro. The levels of NAbs effective against closely related HIV-1 isolates within the same genetic subtype should increase with each additional immunization. Targets of these NAbs will include at least a subset of HIV-1 primary isolates from the same and different viral subtypes. Typically, at least a majority of a panel of primary viruses from at least two different subtypes will be neutralized to at least the 50% level by a 1:10 dilution of the serum that is obtained 2 weeks after the last immunization. The choice of neutralizing assay will not influence the determination of the overall effectiveness of the vaccine, although it is well known that different assays have differing levels of sensitivity and that different viruses have different levels of sensitivity to NAbs. Sera with high activity against multiple subtype primary HIV would be predicted to neutralize most representative primary HIV from many subtypes, regardless of the assay or the choice of primary viruses.

The following examples illustrate representative embodiments now contemplated for practicing the invention, but should not be construed to limit the invention.

### Example 1

This Example describes consistent patterns of change during diversification of the predicted HIV-1 Envelope proteins in SHIV-infected macaques.

### 1. Methods

SHIV89.6P challenge stock: SHIV-89.6P bulk culture (provided by Dr. Norman Letvin, Harvard University) was passaged twice through M. nemestrina PBMCs then used to challenge M. nemestrina intrarectally with 50 times the 50% macaque infectious doses (MID₅₀) (Doria-Rose et al. (2003) J. Virol. 77:11563-77). Sequencing of proviral gp120 from the PBMCs used to grow the virus revealed that the consensus sequence was identical to the published sequence SHIV 89.6P KB9 (accession no. U89134) (data not shown).

PBMC extraction, PCR, cloning and sequencing: All extractions were performed in a separate PCR containment hood using unidirectional workflow, separate storage of templates, primers and other reagents, and other cleaning precautions to avoid sample cross-contamination. DNA was extracted using Qiagen QIAamp DNA Mini Kit and QIAamp DNA Blood Mini Kit according to the manufacturers instructions. Nested PCR was performed on 200 ng to 500 ng genomic DNA in order to amplify proviral V1-C5. To avoid amplification bias of the template, 5 replicates were performed on each sample. Control amplifications with no template were included in each experiment to monitor for carryover contamination. First round PCR was performed with the primers 3′half start (5′ GCATGCTGTAGAGCAAGAAATGGAGC 3′, SEQ ID NO:1) and 1548 (5′ GCTCCCAAGAACCCAAGGAAC 3′, SEQ ID NO:2) using the following cycling conditions: denature at 94° C. for 3 minutes, 35 cycles of 94° C. for 15 seconds, 55° C. for 3 minutes, and 68° C. for 3 minutes, with a 5 second increase on each cycle, followed by a final extension at 68° C. for 7 minutes.

Nested PCR was performed on a 1:25 dilution of first round PCR product using the primers 3′ gp120 89.6 (5′CGATTCATCTTTTTTCTCTTTGCACTGT 3′, SEQ ID NO:3) and EO (5′ TAGAGCCCTGGAAGCATCCAGGAAGTCAGCCTA 3′, SEQ ID NO:4). The following cycling conditions were used: denature at 94° C. for 2 minutes, 25 cycles of 94° C. for 15 seconds, 64° C. for 3 minutes, and 68° C. for 3 minutes, with a 5 second increase on each cycle, followed by a final extension at 68° C. for 7 minutes. PCR product was purified using Qiagen's QIAquick PCR purification kit (Cat No 28104) according to the manufacturers instructions. For each sample, the purified nested PCR products were pooled and ligated into the 2.1 TOPO-TA vector (Invitrogen Cat No 45-0641) using 4 microliters of PCR product and 1 microliter of vector. 2 microliters of ligation mix were transformed into TOP10F′ chemically competent cells (Invitrogen Cat No 44-0300). Transformed cells were selected for with ampicillin and X-gal. 10 clones were obtained from each sample and DNA was prepared using a Qiagen miniprep kit according to the manufacturers instructions. Inserts were confirmed by digestion with EcoRI. Cloned V1-C5 was sequenced using the primer 319 (5′ CATGGTAGAACAGATGCATGAGG 3′, SEQ ID NO:5)) and the TOPO-TA cloning primers M13F and M13R Sequences giving missense predicted proteins were removed from analysis, as were duplicate sequences, resulting in an average of 9 clones analyzed per animal per time point. The proviral env of the inoculating virus was amplified from the PBMC stock that it was prepared in according to the same methods described above. A consensus was created from the eight sequenced clones obtained. The consensus sequence was identical to the published sequence of SHIV 89.6P KB9.

Sequence analysis: Sequences were assembled using Lasergene DNAstar Seqman and Editseq and aligned with ClustalX. Nucleotide alignments were manually edited using BioEdit 5.0.9. A maximum likelihood tree of the nucleotide sequences was constructed with PAUP* 4.0 (Swofford (2003) Phylogenetic Analysis Using Parsimony (* and Other Methods), version 4 ed. Sinauer Assoc., Sunderland, Mass.). For an analysis of diversity, nucleotide alignments were analyzed in MEGA3.0 using the Kimura 2-parameter model with pairwise deletions (transition-to-transversion ratio of 2). For an analysis of divergence, a consensus sequence from eight clones of the inoculating strain was obtained using the LANL Seqpublish program (http://www.hiv.lanl.gov/content/hiv-db/SeqPublish/seqpublish.html). The consensus sequence of the inoculating virus was compared to each clone and divergence was measured with MEGA3.0 using Kimura 2-parameter model with pairwise deletions (transition-to-transversion ratio of 2). The average divergence of each time point for each animal is reported. All sequences were compared to the published sequence to produce the percent difference from the published sequence. All of the clones from the inoculum were then compared to the clones from each animal in order to test whether or not the inoculum is significantly different from the animal's quasispecies using a Mann Whitney test (http://eatworms.swmed.edu/˜leon/stats/utest.cgi). In order to compute selective pressure, nucleotide alignments were compared to the consensus of the inoculating virus using MEGA3.0. Synonymous rates (dS) and Nonsynonymous rates (dN) for each clone were obtained with the modified Nei-Gojobori p-distance model under the following conditions: pairwise deletion, distances only, transition-to-transversion ratio of 2. Synonymous changes are those that alter the DNA sequence of amino acid codons in coding regions without altering the translated amino acid sequence, due to redundancy in the genetic code. Nonsynonymous changes are those DNA changes in codons that result in changes to the amino acid sequence of the protein. The frequency of use for each potential N-linked glycosylation site (PNG) was determined by manually creating a matrix of PNG locations in the software program Microsoft Excel. The nucleotide sequences were translated to amino acids using JavaScript DNA Translator 1.1 (https://deathstar.bcc.washington.edu/public/dnatranslater/index.html), aligned with ClustalX and manually edited with BioEdit 5.0.9. The reference sequence HIV-HXB2 was included in the alignment in order to standardize the numbering positions of each amino acid residue (Korber et al. (1998) in Human Retroviruses and AIDS, vol. III, p. 102-11). Each clone was then scored for the presence or absence of the canonical PNG motif (Asn-X-Ser/Thr where X represents any residue other than proline). The presence or absence of a given PNG motif was then compared to the consensus of the inoculum and is reported as a percentage of use for a given PNG site. The prevalence of a PNG motif within an animals quasispecies was compared to the prevalence of the same PNG motif within the inoculum using a Fisher's exact t-test; p-values <0.05 are reported as significant.

Construction of homologous pseudovirus: In order to test for homologous neutralizing antibodies a single round infection SHIV-89.6P Pseudovirus was utilized. The viral backbone plasmid Q23ΔEnv was kindly provided by Julie Overbaugh (Long et al. (2002) AIDS Res. Hum. Retroviruses 18:567-76). The 89.6P Env plasmid was constructed in PEMC*. The proviral 89.6P gp160 env was first cloned into the 2.1 TOPO-TA vector as described above. First round primers were EO (5′ TAGAGCCCTGGAAGCATCCAGGAAGTCAGCCTA 3′, SEQ ID NO:4) and EO1 (5′, TCCAGTCCCCCCTTTTCTTTTAAAAA 3′, SEQ ID NO:6) and cycling conditions used were: denature at 95° C. for 5 minutes, 35 cycles of 94° C. for 40 seconds, 61° C. for 30 seconds, 72° C. for 3:15 minutes, and a final elongation of 72° C. for 10 minutes. Second round primers were 89.6p 3′ gp41 ClaI (5′ GGCGGCGGCATCGATTCACAAGAGAGTGAGCTCAAGC 3′, SEQ ID NO:7) and 89.6p 5′ gp120 NheI (5′ GCGGCGGCGGCTAGCACAGAAAAATTGTGGGTCACAG 3′, SEQ ID NO:8) and cycling conditions used were: 92° C. for 5 minutes, 40° C. for 1 minute, 68° C. 5 minutes, 92° C. for 1 minute, 45° C. for 1 minute, 68° C. for 5 minutes, 2× (92° C. for 1 minute, 50° C. for 1 minute, 68° C. for 5 minutes), 2× (92° C. for 1 minute, 55° C. for 1 minute, 68° C. for 5 minutes), 25× (92° C. for 1 minute, 60° C. for 1 minute, 68° C. for 5 minutes), 68° C. for 10 minutes. The second round primers inserted an in-frame NheI restriction site 3′ of the env leader sequence and a ClaI restriction site at the 3′ end of gp41. Transformants were generated as described above. The purified plasmid was digested with NheI (Roche Cat No. 885851) and ClaI (Roche Cat No. 404219) and the gp160 fragment was ligated in to PEMC* with T4 DNA ligase (NEB Cat no. M0202S). 5.5 ng total plasmid DNA was transformed in to 50 microliters of MAX Efficiency DH10B Competent Cells (Invitrogen Cat No. 18297-010) as per manufacturers instructions and grown at 30° C. for 24 hours. The plasmid was purified with a Qiagen miniprep kit as described above and sequenced for verification of the insert. 293T cells were transfected with Fugene 6 (Roche Cat No. 11814443001) as per manufacturers instructions. Cells were plated to 50% confluency in a T75; 4 micrograms total DNA with a 2:1 backbone:Env ratio were prepared in a 12 microliterFugene/188 microliter DMEM mixture. Virus was harvested 48 hours later, spun at 2000 RPM for 10 minutes, and stored at −70° C. until use. The pseudovirus was titered on Tzm-bl cells and 200TCID50 were added to each neutralization assay.

Neutralization assay: Sera from each animal were tested at 0, 12, 20, 32, and 45 weeks post infection. Sera were heat inactivated at 56° C. for 1 hour and spun at 14,000 RPM for 5 minutes to remove coagulants. The Tzm-bl neutralization assay was performed in 96-well plates (Wei et al. (2002) Monotherapy Antimicrob. Agents Chemother. 46:1896-1905). Briefly, 200TCID50 virus were added to serial dilutions of sera in the presence of 7.5 micrograms/ml DEAE Dextran and incubated in a total volume of 150 microliters media (DMEM, 10% FCS, 1% L-glutamine, 1% penicillin-streptamycin) for 1 hour at 37° C. Each well received 100 ul of Tzm-bl cells resuspended in media at 1×10⁵ cells/ml. 48 hours later cells were lysed for 2 minutes directly on the neutralization plate using 100 microliters of Bright-Glo Luciferase Assay Substrate (Promega Cat No. E263B) and immediately analyzed for luciferase activity on the luminometer. The inverse dilution of sera necessary to achieve 50% neutralization is reported. Note that the late sera ranges from week 32 (wk32) to week 53 (wk53) but is referred to as week 40 (wk40) for ease of reporting. Neutralization values greater than 3× the prebleed level of background are considered significant. The lowest dilutions of prebleed sera tested (1:100) never reached 50% neutralization; therefore values above 300 were considered significant. Any sample that did not neutralize at 1:100 is reported as 50. All values are calculated with respect to virus only.

### 2. Results

SHIV89.6P-infected Macaca nemestrina: The goal of this study was to examine viral Envelope protein divergence and homologous neutralizing antibodies in a group of macaques that had been challenged with the same virus stock. We performed a vaccine challenge study in M. nemestrina (Doria-Rose et al. (2003) J. Virol. 77:11563-77) using a stock of SHIV-89.6P grown in M. nemestrina cells and titered in vivo. The macaques were challenged with SHIV-89.6P by the intrarectal route, and all were infected, as determined by viral plasma RNA. These animals were monitored for approximately one year. The vaccines used in the study included recombinant vaccinia virus (SHIV-89.6 Gag and Env; termed “Vac” in Table 1), DNA expression vectors (entire SHIV-89.6 genome; termed “DNA” in Table 1) and whole inactivated SHIV-89.6-KB9 treated with AT-2 (termed “part” for inactivated virus particle in Table 1). The vaccines were used in combinations of priming and boosting to compare the efficacy of DNA prime and DNA boost (“DNA-DNA”); DNA priming and vaccinia boosting (“DNA-Vac”); vaccinia priming and DNA boosting (“Vac-DNA”); and DNA priming, inactivated virus particle boosting (“DNA-part”). Statistically significant differences in virus control and CD4⁺ T cell decline were observed in the twelve animals that received DNA plus vaccinia vaccines, and improved outcome was correlated with both cellular and humoral immunity. In contrast, macaques that received DNA prime and either DNA or protein boost did not control viremia nor maintain their CD4+ T cells. Eleven macaques from this study were chosen as representatives from each vaccine group; seven of the animals were unprotected with high viral load set points (greater than 10⁴ RNA copies/ml) and four of the animals were protected with low viral load “set points” (less than 10⁴ RNA copies/ml at 6-12 weeks post-infection), as shown in Table 1.

TABLE 1
Characteristics of SHIV89.6P-infected Macaca nemestrina

| | | | Viral Load | CD4⁺ T cells | | | |
|---|---|---|---|---|---|---|---|
| | | Vaccine | (RNA copies/ml | (copies/microliter | Ratio^{a} | Wk 32^{b} | Wk 32 |
| | Animal | Group | plasma 32 wpi) | 32 wpi) | dN/dS | divergence | diversity |
| Not | K97107 | Control | 2.8 × 10² | 1155 | 1.9 | 0.3% | 0.6% |
| Diversified | K98099 | DNA-Vac | 1.2 × 10³ | 601 | 0.6 | 0.3% * | 0.5% |
| | L97191 | DNA-Vac | 2.5 × 10⁵ | 73 | 1.7 | 0.3% * | 0.5% |
| | J97168 | Vac-DNA | 5 × 10¹ | 1372 | 3.3 | 0.4% | 0.7% |
| Diversified | T98098 | DNA-part | 1.6 × 10⁵ | 40 | 2.2 | 0.9% * | 1.2% |
| | A98069 | DNA-DNA | 1.2 × 10⁴ | 141 | 2.2* | 1.2% * | 1.6% |
| | J98071 | Control | 1.4 × 10⁵ | 29 | 1.9 | 1.4% ** | 1.3% |
| | J97156 | DNA-part | 5.6 × 10⁴ | 56 | 2.0** | 1.8% ** | 1.6% |
| | K97246 | DNA-DNA | 1.6 × 10⁵ | 11 | 2.7** | 1.9% | 1.5% |
| | T98108 | DNA-part | 5.9 × 10⁴ | 5 | 1.8** | 1.9% ** | 1.0% |
| | L98152 | Vac-DNA | 5 × 10¹ | 1820 | 2.8** | 1.9% ** | 1.4% |

^{a}Average ratio within the proviral clones of nonsynonymous change to synonymous change using a modified Nei-Gojobori p-distance. dN was compared to dS using a paired 2-tailed t-test. P values < 0.05 are denoted with *, P values < 0.001 are denoted with **.
^{b}Average percent divergence of each animals cloned quasispecies away from the inoculum. Mann Whitney P values < 0.05 are denoted with *, P values < 0.001 are denoted with **.
The horizontal line divides animals that do not have any significant PNG changes from those that do.
They are ordered by increasing levels of divergence.

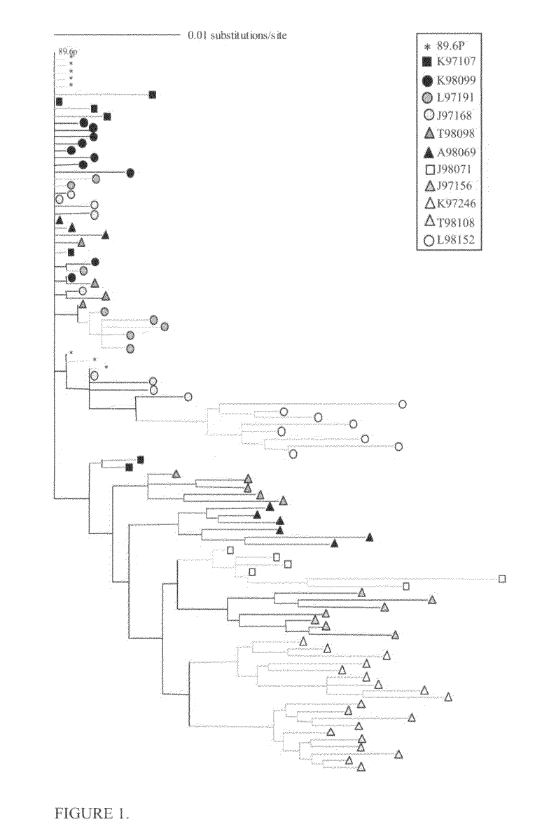

Envelope divergence rates in SHIV-89.6P infection at 32 weeks post infection are similar to HIV-1 infection in humans: Medians of 9 (range 6-10) unique gp120 sequences free of missense mutations were obtained from the proviral sequences of each animal. A maximum likelihood phylogenetic tree was constructed from the gp120 clones obtained at 32 weeks post infection (wpi). The published SHIV-89.6P sequence (Accession No. U89134) and 8 clones obtained from the preparation of the viral inoculum were also included for comparison (FIG. 1).

Further analysis of the aligned nucleotide sequences allowed for quantification of the horizontal distance depicted on the phylogenetic tree. Divergence is defined as viral evolution away from the original sequence while the quasispecies or sequence variation present within an animal at a given time defines diversity. As expected there is only slight heterogeneity (0.3%) within the inoculum. Sequences obtained from four of the eleven animals show little divergence (mean 3.5%, range 0.3%-0.4%) and little diversity beyond that of the inoculum (mean 0.58%, range 0.5%-0.7%). These animals are referred to as “not diversified” or “nondivergent” from here on. In contrast the sequences obtained from each of the remaining seven animals show marked divergence (mean 1.5%, range 0.9%-1.9%) and diversification (mean 1.4%, range 1.1%-1.6%) These animals are referred to as “diversified” from here on (Table 1). The average divergence of all 11 animals at 32 wpi is 1.1%, giving an average rate of 1.8% divergence in gp120 per year. This is similar to the 1% per year rate of divergence reported in HIV-1-infected patients (Shankarappa et al. (1999) J. Virol. 73:10489-502.

There are several clones within the four nondivergent macaques and in the inoculum that have a limited number of unique changes from the consensus sequence of SHIV-89.6P. These are represented as individual short terminal branches in the phylogenetic analysis. In contrast, the seven macaques with divergent sequences each form a unique branch. This supports the conjecture that the clustered sequences were derived from the same macaque and that each quasispecies is unique to an animal. None of the sequences obtained from the inoculum cluster with any of the seven animals shown to diverge, suggesting that the divergence does not represent the common outgrowth of a minor sequence.

As a measure of selective pressure the rate of nonsynonymous to synonymous change was calculated for each clone. Each clone was compared pairwise to the consensus of the inoculum using a modified Nei-Gojobori p-distance to calculate dN and dS. These values were analyzed using a paired 2-tailed t-test to determine if the nonsynonymous rate of change was significantly different than the synonymous rate of change within the quasispecies. The ratio of dN/dS is reported in Table 1 (*=<0.05, **=<0.001). None of the non-diversified animals had significant ratios of dN/dS while five of the diversified animals had a significantly higher dN than dS (Table 1).

Higher viral load set point is associated with higher levels of Env divergence: Six of the seven macaques with high viral load set points (greater than 10⁴ RNA copies/ml) developed quasispecies that diverged from the inoculum (Table 1). The one exception is animal L97191, which has the highest viral load set point but whose viral population is 0.3% divergent from the inoculum at 32 wpi. In contrast, only one of the four animals with low viral set points (less than 10⁴ RNA copies/ml) developed quasispecies that diverged from the inoculum. Similarly the control animals and those that received ineffective vaccines (DNA/DNA or DNA/protein) tended to be the animals with higher viral loads and therefore greater divergence of their quasispecies. This trend does not reach significance (p value=0.09).

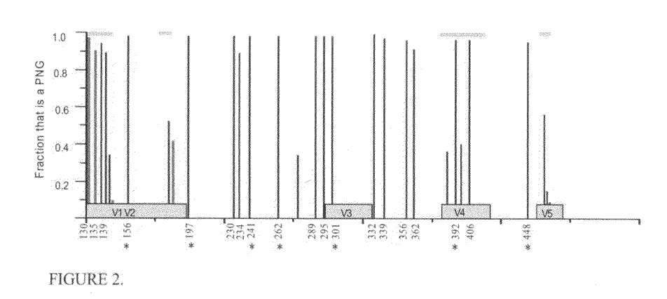

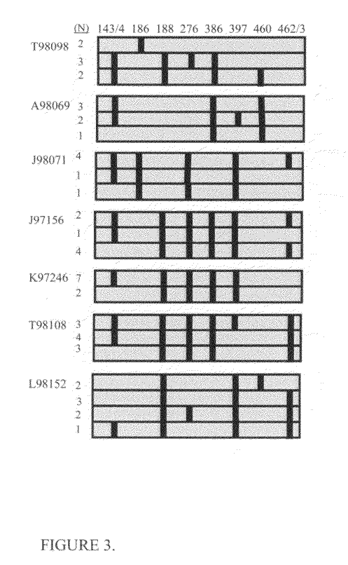

Many predicted glycans are unchanged in gp120, regardless of level of divergence: The SHIV-89.6P inoculum contained 23 PNGs on gp120. Nineteen were present at the same location in over 90% of all sequences analyzed as a group. Seven of the highly conserved PNGs were located within the variable loops, suggesting that the positions of these glycans are highly advantageous if not necessary for the function of the virus. These sites include four PNGs in V1 (N130, N135, N139, N156), one site in V3 (N301), and two sites in V4 (N392, N406) (FIG. 2). Modeling these residues on to the gp120 core structure of HIV-1 (Kwong et al. (1998) Nature 393:648-59) demonstrates that many of these well-conserved PNGs map to the outer domain, or silent face (Wyatt et al. (1998) Nature 393:705-11) of gp120 (FIG. 3). When the quasispecies of each macaque were compared individually, eleven of the PNGs described above were found to be well conserved in all of the macaques tested. Four of the highly conserved PNGs were located within the variable loops: V1 (N156), V3 (N301), and V4 (N392, N406). The remaining sites were at N197, N230, N241, N289, N295, N332, and N339.

The locations of these conserved PNGs are closely similar to conserved PNGs in Envs from patients in the Los Alamos National Laboratory database, when a broader cross sectional analysis of 94 published full length Env sequences from Clades A, B, and C are analyzed in the same manner. HIV gp120 has between 18-33 PNGs (Zhang et al. (2004) Glycobiol. 14:1229-46). In this cross sectional analysis, 11 PNG sites were found to be present in over 90% of all sequences analyzed. Of those, seven PNG sites were identical to those highly conserved in the SHIV-89.6P dataset (N156, N197, N241, N262, N301, N392, N448) and included 4 sites within variable regions (FIG. 2). This result supports the hypothesis that lentiviral Envelopes maintain a core set of glycans that are important in producing a functional protein regardless of the HIV or SHIV background.

Convergent PNG changes occur in the quasispecies of diverged Envs from independent macaques: We found four PNG additions and two PNG shifts that occur independently in multiple macaques with divergent quasispecies. As described above many of the glycans were well conserved. Functionality of the Env protein appears to limit the number of glycans, also termed “sequons” that can be added (Zhang et al. (2004) Glycobiol. 14:1229-46). Therefore the remaining glycans are free to vary their positions, though the net number does not increase. Although some glycan changes are unique to the variant and host, multiple changes occurred in remarkably consistent ways across different macaques. For each animal the prevalence of a given PNG location within that animals quasispecies is reported as a percentage that is compared to the prevalence of the same PNG within the inoculum. In Table 2, we show the percentages for each PNG location that underwent a significant level of change in more than one animal (Fisher's exact t-test, p-value <0.05). In four of the seven divergent animals a PNG was added in V1 at either N143 or N144. There are two PNG additions on the N terminal side of V4: N386 is added in 5/7 animals while N397 is added in 4/7 animals. The PNG in the outer region of C2, N276, has been defined as part of the CD4 binding site (Kwong et al. (1998) Nature 393:648-59). In the HIV cross sectional analysis of 94 published full length Env sequences, N276 is seen to be present in 98% of the sequences.

TABLE 2
Convergent PNG changes in gp120 at 32 wpi^{a}

| | | Add | Shift | | Add | Add | | Shift | |
|---|---|---|---|---|---|---|---|---|---|
| | | V1 | V2 | | C2 | V4 | | V5 | |
| | (N)^{b} | 143/4 | 186 | 188 | 276 | 386 | 397 | 460 | 462/3 |
| Inoculum^{c} | 8 | 0% | 100% | 0% | 0% | 0% | 38% | 100% | 0% |
| K97107 | 8 | 25% | 100% | 25% | 0% | 0% | 0% | 100% | 0% |
| K98099 | 10 | 0% | 100% | 0% | 0% | 0% | 0% | 90% | 0% |
| L97191 | 10 | 0% | 100% | 0% | 0% | 0% | 0% | 100% | 0% |
| J97168 | 10 | 0% | 89% | 0% | 0% | 0% | 33% | 100% | 0% |
| T98098 | 9 | 55% | 44% | 56% | 33% | 56% | 0% | 44% | 0% |
| A98069 | 10 | 56% | 33% | 0% | 0% | 67% | 22% | 100% | 0% |
| J98071 | 9 | 83% | 100% | 0% | 100% | 17% | 100% | 0% | 67% |
| J97156 | 8 | 43% | 0% | 100% | 100% | 100% | 100% | 0% | 86% |
| K97246 | 10 | 78% | 0% | 100% | 100% | 100% | 100% | 0% | 0% |
| T98108 | 10 | 70% | 0% | 100% | 100% | 100% | 30% | 0% | 100% |
| L98152 | 10 | 13% | 0% | 100% | 25% | 0% | 100% | 25% | 75% |

^{a}Percent prevalence of each PNG within the quasispecies. Only sites that differ significantly from the inoculum in more than one animal are shown. Bolded numbers indicate a Fisher's Exact P value < .05. The horizontal line divides animals that do not have any significant PNG changes from those that do. They are ordered by increasing levels of divergence.
^{b}Ten proviral clones were analyzed from each animal. Duplicate or missense sequences were removed from analysis. N represents the number of clones incorporated for analysis for each animal.
^{c}Proviral clones from the PBMCs used to grow the viral stock of SHIV-89.6P were analyzed as a measure of heterogeneity in the inoculum.

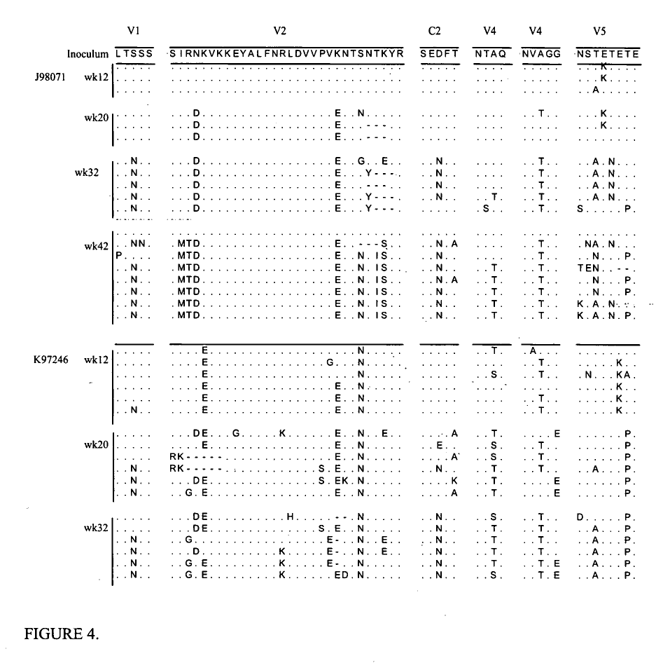

This site was not present in the SHIV-89.6P infecting virus used in this study but was added in four of the seven divergent macaques. A PNG shift is being defined as the removal of one PNG with the concurrent addition of a new PNG within 2 to 3 residues. There is a shift that occurs in V2 from N186 to N188 in 5/7 divergent animals. This shift is complete (occurring in 100% of the clones) in four of the animals. There is another shift that occurs in four of the seven macaques in V5 from N460 to either N462 or N463 (Table 2) Although these six PNG changes are common to multiple animals, identical combinations of these 6 PNG changes occur only in a few macaques (J97156 shares one type with K97246, one type with T98108), all with differing amino acid sequences surrounding these sequons (FIG. 4 and data not shown). These data further support the concept that changes represent convergent evolution rather than the outgrowth of a minor variant (FIG. 3). The cross sectional analysis of full length HIV Env also revealed preferential regions of PNG change, or hotspots. As described above the location of approximately half of the PNGs were well conserved. Of those PNGs with variable positions, the majority of change mapped to limited windows of the variable regions. These hotspots include the N terminal side of V1, a small region on the C terminal side of V2, V4, and a small region of V5, depicted schematically in FIG. 2. Five of the 6 consistent PNG changes in the SHIV-89.6P model fall within these hotspots. Modeling these residues on to the gp120 core structure of HIV-1 demonstrates that four of the consistent PNG changes are proximal to the predicted CD4 binding site (CD4bs) (Kwong et al. (1998) Nature 393:648-59). The remaining two changes are seen in V1V2 and can therefore not be modeled in this manner.

The viral quasispecies from a subset of macaques were analyzed longitudinally and show the steady accumulation of consistent changes over time (FIG. 4). This analysis showed that in the macaques studied, changes did not occur together temporally. This is demonstrated in the quasispecies of macaque K97246 which undergoes a complete PNG shift in V2 from N186 to N188 by 12 wpi, while the addition of N276 in C2 does not occur until 32 wpi (FIG. 4). These changes also occur at different rates in each macaque. For example, the addition of N386 occurs in some clones at week 12 earlier in macaque K97246 while it is only seen in one clone at week 32 in macaque J98071.

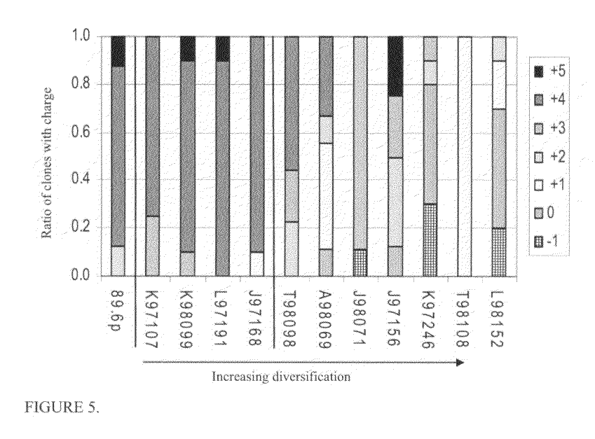

Changes in charge and potential changes in structure are conserved in multiple macaques: Although the majority of common sequence changes occur at PNG sites, we observed two additional types of changes in the hypervariable regions. There is a 40-residue stretch of V1V2 that has a strong net positive charge (+5) in the consensus sequence of the SHIV-89.6P virus used to challenge the macaques. In the cloned inoculum 88% of the sequences have a net charge of +4 or +5. The four animals with viral sequences that do not diverge from the inoculum share this charge profile (89% have a net charge of +4 or +5). In all seven macaques with divergent quasispecies, this region of V1V2 becomes more acidic by 32 wpi, with only 15% of the clones having a net charge of +4 or +5 (FIG. 5). A proline is added to V5 by 32 wpi in five of the seven macaques with divergent quasispecies (mean 66%, range 38% to 100%). This mutation occurs at either residue 462 or 465. This region of V5 is flanked by residues that are predicted to be involved in the CD4bs (Kwong et al. (1998) Nature 393:648-59).

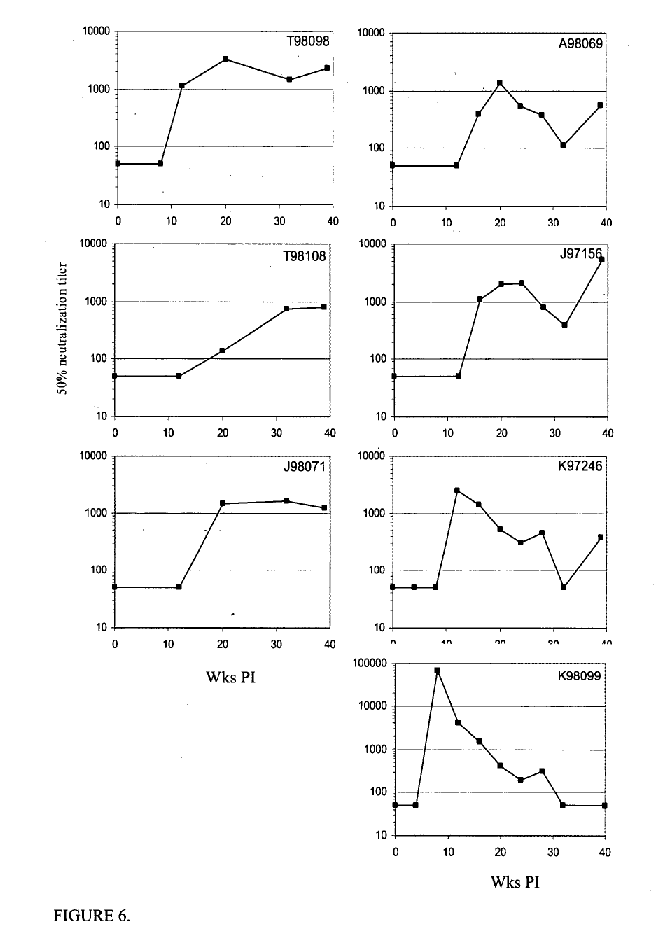

Macaques with divergent Env quasispecies have higher levels of homologous neutralizing antibodies: Although none of the macaques in this study had detectable NAbs at the time of challenge, one macaque was a member of a vaccine group with accelerated NAbs post-challenge. In the remaining macaques, the development of a divergent quasispecies correlates with the slow and sustained development of homologous NAbs (FIG. 6).

Three of the four non-divergent macaques never develop detectable levels of homologous neutralizing antibodies within the one-year span of this study (K97107, L97191 and J97168). Macaque K98099 showed no divergence from the inoculum yet rapidly developed homologous NAbs by week 12, the highest titer of any in this study. This response also declined rapidly, showing a weak response by week 20 and undetectable response by week 32. As is characteristic of the nondivergent animals, this animal has a low viral load set point, likely as a result of the DNA/Vaccinia vaccine that it received (Doria-Rose et al. (2003) J. Virol. 77:11563-77).

Of the macaques with divergent quasispecies, 6 of the 7 develop homologous NAbs. By 12 wpi, plasma from two macaques achieved appreciable levels of homologous NAbs: K97246 and T98098. An additional 3 animals develop homologous NAbs by 20 wpi: A98069, J97156, and J98071. The remaining animal, T98108, began to develop a low titer homologous NAb response by week 32. All six macaques maintained a homologous response through the latest time point tested. Three of the macaques (J98071, T98108, and T98098) maintain steady titers of homologous NAbs. J98071 is a control animal that developed a homologous response at 20 wpi that is maintained through 45 wpi. Macaque T98098 received a DNA/protein vaccine and developed a homologous response by 12 wpi that was maintained through 45 wpi. T98108 also received a DNA/protein vaccine but did not develop a detectable homologous response until 20 wpi. The other three animals (A98069, J97156, and K97246) developed strong homologous NAbs at 20, 20, and 12 wpi respectively but showed a declined titer at 32 wpi. The titer of homologous NAbs then continued to increase in all three macaques by week 45. The one divergent animal that did not develop homologous neutralizing abs (L98152) is also unusual in that it is the only divergent animal with a low viral load.

### 3. Discussion

The high degree of variability that is tolerated by the HIV Envelope coupled with the viruses extensive use of carbohydrates as a means of neutralization escape has contributed to the formidable challenge of developing an affective vaccine against HIV. In recent years a great deal of insight has been gained from structural studies of the liganded and unliganded gp120 core (Chen et al. (2005) Nature 433:834-41; Kwong et al. (1998) Nature 393:648-59) as well as several studies looking at the dynamics of the glycan shield in both SIV (Chackerian et al. (1997) J. Virol. 71:7719-27) and HIV (Cheng-Mayer et al. (1999) J. Virol. 73:5294-5300; Choisy et al. (2004) J. Virol. 78:1962-70; Dacheux et al. (2004) J. Virol. 78:12625-37; Wei et al. (2003) Nature 422:307-12). Such studies have documented that the arrangement of carbohydrates can allow for neutralization escape mutants while many of the individual glycans play important roles in protecting the CD4 binding site and pg41 ectodomain from neutralization antibodies (McCaffrey et al. (2004) J. Virol. 78:3279-95). A major question that remains unanswered lies in the boundaries of variation that exist as the virus evolves in individual hosts, a question that has been difficult to address in HIV-1 infection of humans due to the distinct viruses in each individual. One advantage that the nonhuman primate model is that the same challenge virus can be administered to multiple outbred hosts, providing a uniform starting point from which to examine whether any inherent patterns emerge in the way that the virus evolves in concert with host immune pressure. This study of longitudinal and cross-sectional mutations in HIV Env in the SHIV background shows that there is more constraint on the configuration of the glycan shield than was previously appreciated and that mutations are clustered in what we are terming “hotspots.” A cross-sectional analysis of published HIV Env sequences from multiple subtypes demonstrated (Zhang et al. (2004) Glycobiol. 14:1229-46) that there are key regions that are seen to vary. In this analysis of the SHIV-89.6P Env in vivo, we have observed that several of the trends seen in the cross section human study are seen in this nonhuman primate study.

A striking result of this analysis was that only a small percentage of predicted N-linked carbohydrate attachment sites were subject to significant variation in any of the macaques; after 32 weeks of infection 19 out of 23 PNGs still maintained the same position in over 90% of all sequences. In several cases this held true even when the PNGs were located in variable regions. Several of these PNGs are also well conserved at the same locations in the HIV data. In addition, the changes that did occur were remarkably consistent between animals. In particular four PNGs were commonly added and two PNGs were commonly shifted. When considering these 6 PNG changes it is noteworthy that only three of the seven animals with divergent virus had exactly the same combination of PNG changes, each in different sequence backgrounds. This suggests that the sequon changes were arrived at in an independent manner and did not arise from the outgrowth of a minor variant. There is a scattering of unique PNG changes that occur in only one animal or at low levels. The changes that occur in only one animal may reflect positions that are not vital to but are generally favored for the function of the virus. Others have documented examples where a specific PNG was less important then the density of PNGs in a given region; the unique PNG changes seen in this model may lend further evidence to this phenomenon (Ohgimoto et al. (1998) J. Virol. 72:8365-70; Quinones-Kochs et al. (2002) J. Virol. 76:4199-4211). It is also possible that these scattered changes represent nonviable virus, as the sequences analyzed in this study were obtained from provirus. Nevertheless the six PNG changes that occur significantly in multiple animals documented here are likely to represent viable changes in the virus since it is unlikely that such nonviable changes would be so pronounced in multiple animals due to chance alone.

The CD4bs is a conformational determinant of Env that is conserved among the primate lentivirus family. At least some of the broadly neutralizing activity in human serum has been shown to map to this region (Steimer et al. (1991) Science 254:105-8), and human mAbs have confirmed that at least some antibodies such as IgG-1b 12 targeted to this region can neutralize divergent isolates (Burton et al. (1994) Science 266:1024-7). It is thought that the coreceptor binding site is only available after conformational changes induced by CD4 and that the gp41 ectodomain is only available after conformational changes induced by coreceptor binding (Chen et al. (2005) Nature 433:834-41). Therefore it could be argued that the CD4bs is the most vulnerable region of the viral Env, and thus mutations that shield it from attack by antibodies would provide a fitness advantage to the virus. This need to protect the CD4bs from neutralizing antibodies may come at a price of decreased optimization for CD4 binding, as evidenced by the tendency for lab-adapted viruses free from immune pressure to evolve a more open conformation of the CD4bs (Moore & Ho (1995) AIDS 9:S117-36). It is thought that the V1V2 and V3 loops overlap and form a protective barrier around the CD4 binding site (D'Costa et al. (2001) AIDS Res. Hum. Retroviruses 17:1205-9; Zwick et al. (2003) J. Virol. 77:6965-78). The V3 loop is thought to play the predominant role in physical interactions with the coreceptor and has a strong net positive charge that is able to bind to the negatively charge CCR5 coreceptor. Due to this requirement the polarity of the V3 loop is relatively constrained. However if the V1V2 loops adopt a more negative charge, this could influence the packing of the variable loops around the CD4bs (Etemad-Moghadam et al. (1998) J. Virol. 72:8437-45; Poss et al. (1998) J. Virol. 72:8240-51). In this study all seven animals with divergent quasispecies evolved a more acidic V1V2. In addition to changes in glycosylation, this change in the net charge of V1V2 may reflect a common mechanism for protecting the CD4bs when faced with immune pressure. However, this mutation was also seen in one macaque with low virus and diminishing NAbs, suggesting that the mechanism may not be involved in NAb development.

Little is known about how the V5 region changes over time. Both the N- and C-terminal amino acids in the V5 loop are known to comprise part of the CD4bs. The proline change seen in this study would be predicted to change the shape of the V5 loop and may in turn alter the CD4bs. The three-dimensional structure predictions show that residues in V5 are close to the CD4bs.

A major goal of the study was to determine how nonsynonymous changes in Env were related to the development of homologous and heterologous NAbs. The present study utilized a subset of thirty macaques infected with SHIV-89.6P that were part of a prime-boost vaccine study. We chose to study this group of macaques because they were all infected by the mucosal route following intrarectal challenge with the same viral stock, and they had differing outcomes, depending upon the vaccine regimen employed. The different vaccine groups in this study had demonstrated various levels of effectiveness in controlling viremia, which we showed previously was correlated both with cellular responses and early development of NAbs effective against the challenge virus (Doria-Rose et al. (2003) J. Virol. 77:11563-77). Although the DNA/Vaccinia and Vaccinia/DNA groups controlled viremia well, the DNA/protein and DNA/DNA groups fared no better then the unvaccinated controls. Macaques with high virus loads, independent of vaccine or control group, were predominantly the animals that underwent divergence of viral Env. Importantly, these animals were also the ones to develop persistent homologous NAbs. The one exception was macaque K98099 who demonstrated a strong and rapid homologous neutralizing antibody response. This animal received Vaccinia/DNA prior to challenge and was able to control viremia and maintain CD4. The rapid specific response in this animal was likely the result of a memory response generated from the vaccine. Since viremia was controlled at a low level there was little antigen present in this macaque, which we speculate resulted both in the rapid decline of homologous neutralizing antibodies and little to no diversification of the viral Env.

Our conclusions from this work are that the viral Env diversification is likely to be contributing to increases in titer of homologous NAbs. This question is one that we are in the process of exploring, utilizing the cloned Envs to make pseudovirions and to determine the neutralization profiles of each variant. We do not have evidence for broadening of the NAb response in the one-year time frame of the study. This result is consistent with studies in humans infected with HIV-1, who do not typically develop heterologous NAbs prior to three years of infection (Moog et al. (1997) J. Virol. 71:3734-41; Richman et al. (2003) Proc. Natl. Acad. Sci. U.S.A. 100:4144-9).

Data from this study supports the Nowak model of viral evolution that states that high viral loads are the driving force behind viral diversification. Importantly, the HIV Env data in this study show a consistent relationship with SIV Env data between higher diversification and higher levels of neutralizing antibodies (Hirsch et al. (1998) J. Virol. 72:6482-9). Conversely the HIV data has shown that slower progression often correlates with higher levels of viral diversification as well as more robust neutralizing antibodies (Cao et al. (1995) N. Engl. J. Med. 332:201-8; Delwart et al. (1997) J. Virol. 71:7498-508; Wolinksy et al. (1996) Science 272:537-42). Although at face value these models seem to contradict, one possible explanation is simply a definition of progressor and non progressor status. Macaques with divergent Env in this study were those that failed to prevent the early, rapid CD4⁺ T cell loss that is the hallmark of SHIV-89.6P infection, but even among the vaccine control group, only one of the six macaques was a rapid progressor and died during the study period of one year. Therefore, the macaques in this study could be described as slow or slower progressors. There is a precedent for viral replication driving antibody maturation that was established with the influenza vaccine (Brokstad et al. (1995) Vaccine 13:1522-8). It is interesting to note that several of the animals maintain steady virus loads and that the homologous neutralizing antibodies are either maintained or continue to increase despite the changes occurring in the viral Env. However, further work is necessary to determine whether any of the mutations that we have sequenced encodes an escape variant. In these macaques, the majority of homologous neutralizing antibodies may be directed against the immunodominant V3 loop, which does not undergo change in the time frame of this study. It is possible that the mutations in Env sequences are contributing to the development of higher avidity NAbs or to NAbs effective against heterologous isolates. This study ended after one year of observation, at which point none of the plasma samples showed significant evidence for heterologous neutralization.

There is precedence for convergent patterns of change as HIV evolves within independent hosts. It is well documented that the HIV Clade B viruses often undergo defined changes as the virus switches from an R5 to an X4 phenotype. Hirsch has shown in the SIV model that convergent changes in the viral Env can affect viral fitness by leading to a CD4 independent virus (Dehghani et al. (2003) J. Virol. 77(11):6405-18). These examples highlight patterns in the way that the virus evolves to enhance viral fitness. Data from this work demonstrate additional mechanisms of convergent change in the viral Envelope that involve the glycan shield, V1V2 charge, and V5 structure. These changes correlate with both higher viral load and higher homologous neutralizing antibodies. Further work is needed to define whether these convergent changes affect viral fitness or viral escape. As we continue to deepen our understanding of the intricacies of HIV evolution, we may be able to better inform the design of effective vaccines able to generate the elusive broadly reactive neutralizing antibodies.

### Example 2

This Example describes a representative vaccination protocol according to some embodiments of the invention.

Table 3 provides four examples of combinations of glycosylation mutants that may be used to vaccinate a human host with a series of HIV-1 Env variants in order to optimize the development of homologous and heterologous NAbs. The human host may already infected be with HIV-1, may become infected during the vaccination protocol, or may remain uninfected throughout the entire vaccination protocol. The exact timing of delivery of each component can be varied. In the examples below, only DNA and protein are used. An exemplary regimen for delivery is weeks 0, 12, 20, 32, 34, and 40. The four experimental groups are compared for optimal development of anti-Env immunity: (1) Clonal—Early Env; (2) Clonal—All Changes; (3) Quasispecies Members as Sequential Vaccination; and (4) Quasispecies combination. In Table 3, “Clonal—Early Env” refers to four inoculations, each with a single DNA immunogen having the PNG changes observed in the first few days/weeks of infection (for example, the changes described in EXAMPLE 1), followed by two inoculations with a single protein immunogen having all the changes observed early in infection (for example, the changes described in EXAMPLE 1); “Clonal—All Changes” refers to four inoculations, each with a single DNA immunogen having all the changes observed late in infection (e.g., the six consistent PNG changes, the increase in acidity in the 40-residue stretch of V1V2, and/or the addition of a proline to V5 at position 462 or 463, as described in EXAMPLE 1) followed by two inoculations with a single protein immunogen having all the changes observed late in infection (for example, the changes described in EXAMPLE 1), “Quasispecies Members as Sequential Vaccination” refers to a series of four inoculations, each with multiple DNA immunogens including all or most of the quasispecies members observed at each of the time points after infection (for example, the changes described in EXAMPLE 1) followed by two inoculations with a single protein immunogen having all the changes observed late in infection (for example, the changes described in EXAMPLE 1); and “Quasispecies Combination” refers to a series of four inoculations, each with multiple DNA immunogens including most or all the variants (for example, 16 variants) observed at each time point after infection (for example, as described in EXAMPLE 1) followed by two inoculations with a single protein immunogen having all the changes observed late in infection (for example, the changes described in EXAMPLE 1). The term “changes observed late in infection” refers to changes that found after approximately one year of infection in env genes that have diversified by at least 1% from the initial infecting virus.

TABLE 3
Exemplary Vaccination Protocols

| | | | Quasispecies | |
|---|---|---|---|---|
| | | | Members as | |
| Group | Clonal - Early | Clonal - All | Sequential | Quasispecies |
| Inoculation | Env | Changes | Vaccination | Combination |
| Wk 0 gp160 | Early Env | All changes, | Early Env, n = 1 | All variants |
| DNA | | n = 1 | | each time, n = 16 |
| Wk 12 gp160 | Early Env | All changes, | Wk 12 variants, | All variants |
| DNA | | n = 1 | n = 5 | each time, n = 16 |
| Wk 20 gp160 | Early Env | All changes, | Wk 20 variants, | All variants |
| DNA | | n = 1 | n-5 | each time, n = 16 |
| Wk 32 gp160 | Early Env | All changes, | Wk 32 variants, | All variants |
| DNA | | n = 1 | n = 5 | each time, n = 16 |
| Wk 34 | Early Env | Late protein - | Late protein - | Late protein - |
| Oligomeric | Protein | all changes | all changes | all changes |
| gp140 | | | | |
| Wk 40 | Early Env | Late protein - | Late protein - | Late protein - |
| Oligomeric | Protein | all changes | all changes | all changes |
| gp140 | | | | |

### Example 3

This Example describes a representative vaccination protocol according to some embodiments of the invention.

In this example, the dominant variant that is shown to be an escape mutant from NAbs at the concurrent time of infection is used as the major immunogen for that time point. The human host may already infected be with HIV-1, may become infected during the vaccination protocol, or may remain uninfected throughout the entire vaccination protocol. The exact timing of delivery of each component can be varied. In the examples below, only DNA and protein are used. An exemplary regimen for delivery is weeks 0, 12, 20, 32, 34, and 40.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A vaccination protocol comprising administering to a primate host a first immunogen comprising at least one Human Immunodeficiency Virus type 1 Envelope (env) sequence having a first set of consensus glycosylation sequences, followed by a second immunogen comprising at least one primate immunodeficiency virus env sequence having a second set of consensus glycosylation sequences, wherein the differences between the first set of consensus glycosylation sequences and the second set of consensus glycosylation sequences comprise at least one of:
(a) an addition of a consensus N-linked glycosylation sequence in V1 at a position corresponding to position 143 or 144 in the HIV-89.6 env sequence;
(b) a shift of a consensus N-linked glycosylation sequence in V2 from a position corresponding to position 186 in the HIV-89.6 env sequence to a position corresponding to position 188 in the HIV-89.6 env sequence;
(c) an addition of a consensus N-linked glycosylation sequence addition in C2 at a position corresponding to position 276 in the HIV-89.6 env sequence;
(d) an addition of a consensus N-linked glycosylation sequence addition in V4 at a position corresponding to position 386 in the HIV-89.6 env sequence;
(e) an addition of a consensus N-linked glycosylation sequence addition in C2 at a position corresponding to position 397 in the HIV-89.6 env sequence; and
(f) a shift of a consensus N-linked glycosylation sequence in V5 from a position corresponding to position 460 in the HIV-89.6 env sequence to a position corresponding to position 462 or 463 in the HIV-89.6 env sequence.
